(19) United States
(12) Patent Application Publication
(10) Pub. No.: US 2004/0115800 A1
(43) Pub. Date: Jun. 17, 2004

(54) Selectable marker for genetically engineered cells and tissues

(76) Inventors: Richard L Momparler, Outremont (CA); Jacques Galipeau, Town of Mount-Royal (CA); Nicoletta Eliopoulus, Montreal (CA)

Correspondence Address: KLAUBER & JACKSON, 411 HACKENSACK AVENUE, HACKENSACK, NJ, 07601

(21) Appl. No.: 10/466,705

(22) PCT Filed: Jan. 16, 2002
(86) PCT No.: PCT/CA02/00042

(51) Int. Cl. C07H 21/04; C12N 15/00; C12N 15/09; C12N 15/63; C12N 15/70; C12N 15/74; C12N 5/00; C12N 5/02; C12N 5/08

(52) U.S. Cl. 435/320.1; 536/23.5; 435/325; 435/366

(57) ABSTRACT

The present invention relates to selectable marker gene, method of producing genetically modified cells allowing expression of a bicistronic vector, and ex vivo gene therapy applications. The invention also relates to selection of genetically engineered cells from the mixed population, prior to implantation in vivo. The invention describes the human cytidine deaminase (CD) gene as an ex vivo dominant selectable marker in gene-modified primary cells with cytosine nucleoside analogues. A bicistronic retrovector comprising the human CD coding sequence and the enhanced green fluorescent protein (GFP) reporter gene is used for the transduction of cells. The invention describes the introduction of CD gene in cell and gene therapy applications for the ex vivo dominant selection of genetically engineered cells.

### BACKGROUND OF THE INVENTION

(a) Field of the Invention

The invention relates to novel expression vector for selection of transduced and/or transfected cells expressing gene of interest. The invention particularly relates to a selectable marker gene for genetically engineered cells. Also disclosed is a method of selecting transduced and/or transfected cells for ex vivo gene therapy.

(b) Description of Prior Art

High gene transfer efficiency and transgene expression in target primary cells is an essential but challenging aspiration in gene therapy applications. A remedy may be to dominantly select and enrich ex vivo the cells concomitantly expressing the therapeutic gene and an attached selectable marker gene.

Numerous investigators have employed dominant selection strategies utilising drug resistance genes, such as the prokaryotic neomycin phosphotransferase II (Neo^{R}) drug resistance gene, the human multidrug resistance (MDR) gene, the mutated human dihydrofolate reductase (DHFR) gene, and the human O⁶-methylguanine-DNA-methyltransferase (MGMT) gene. However, disadvantages have been noted with some drug selectable markers. For instance, cells modified with the prokaryotic Neo^{R} gene may elicit an immune reaction in vivo, MDR-transduced hematopoietic stem cells, when transplanted in mice, will lead to a myeloproliferative syndrome and MGMT genetically engineered cells require selection with the DNA damaging alkylating agents. Several investigators have revealed the utility of bone marrow stromal cells, the progenitor cells for nonhematopoietic tissues, in cell and gene therapy strategies. These autologous cells, which are easily isolated from bone marrow aspirates, expanded in culture, and gene modified, have been genetically engineered for the expression of an exogenous gene product and/or for the secretion of therapeutic proteins in vitro and in vivo.

Numerous investigations have reported the use of drug resistance genes for in vivo selection of genetically altered cells. The disadvantages of in vivo drug selection, such as the systemic side-effects of chemotherapeutic agent administration, are not encountered with in vitro drug selection, since enrichment of the population of strong transgene expressing cells is conducted prior to transplantation.

Gene transfer studies regularly employ drug resistance genes for the in vitro selection of genetically engineered cells. For instance, the neomycin phosphotransferase II (Neo^{R}) drug resistance gene confers to transduced cells the ability to be selectively expanded through treatment with neomycin or its analogue G418. The Neo^{R} gene however is not a suitable selectable marker for clinical applications since it is prokaryotic and immunogenic. Moreover, one study reported the adverse effect of the Neo^{R} gene on the concomitant expression of a second gene product (Apperley et al., 1991, Blood 78:310-317).

The human multidrug resistance gene (MDR) which codes for the multidrug transporter P-glycoprotein has been extensively described and utilised as a dominant selectable marker with MDR-responsive chemotherapeutic agents, such as paclitaxel and colchicine (Licht et al., 2000, Gene Therapy 7:348-358). Nonetheless, some investigators observed a myeloproliferative syndrome in mice transplanted with MDR-transduced hematopoietic stem cells.

Another drug resistance gene, the O⁶-methylguanine-DNA-methyltransferase (MGMT) gene codes for a DNA repair enzyme that when overexpressed renders normal mammalian cells resistant to O⁶-alkylating agents, such as the nitrosoureas and related methylating compounds. Since these antineoplastic drugs produce DNA damage which can have toxic, mutagenic, transforming, and carcinogenic repercussions, their use for dominant selection of gene-modified cells may be hazardous.

Alternative dominant selectable markers that are well-defined and widely utilised comprise the mutant variants of the human dihydrofolate reductase (DHFR) gene which maintain a role in folate metabolism when introduced into cells but confer drug resistance against the cytotoxicity of antifolates, such as methotrexate and trimetrexate. Several investigators have accomplished in vitro drug selection of mutant DHFR-transduced cells. However, for the untransduced cells to be eradicated by antifolate toxicity, depletion of thymidine from the cell media is required and may be achieved through thymidine phosphorylase treatment of the media or by the utilisation of dialysed thymidine free serum. Moreover, one study demonstrated that retroviral transfer of the human aldehyde dehydrogenase class-I gene permits in vitro selection with 4-hydroperoxy cyclophosphamide of transduced K562 leukemia cells (Moreb et al., 1998, Human Gene Therapy 9:611-619). Other selectable markers in transfected or transduced cells include the glutamine synthetase gene, the hygromycin resistance gene, the zeocin resistance gene, as well as the puromycin, histidinol D, and phleomycin drug resistance genes.

Retroviral transfer of a selectable drug resistance gene may in some cases confer lower expression of the therapeutic transgene or raise an immune reaction against gene-modified cells, ultimately reducing the effectiveness of the gene therapy approach. The immunogenicity of certain drug selectable genes such as Neo^{R}, led to their removal.

It would be highly desirable to be provided with an efficient selectable marker gene which allow for preparing transduced or non-immunogen transfected cells producing a protein of interest, and for ex vivo gene therapy and protein delivery.

### SUMMARY OF THE INVENTION

An object of the present invention is to provide an expression vector for identifying transduced and/or transfected cells.

In accordance with the present invention, there is provided an expression vector for identifying transduced and/or transfected cells expressing a nucleotitic sequence of interest. The expression vector comprises:

a suitable promoter compatible with the transfected cells;

an internal ribosome entry site (IRES);

a marker nucleotidic sequence encoding drug resistance cytidine deaminase (CD), a functional fragment or an analog thereof, operably linked to the IRES; and

an endogenous or exogenous nucleotidic sequence of interest operably linked to the IRES;

wherein the marker nucleotidic sequence when detected in cells, indicates that the cells are transduced and/or transfected and capable of expressing the nucleotidic sequence of interest, the nucleotidic sequence encoding drug resistance cytidine deaminase (CD), a functional fragment or an analog thereof, being operably linked upstream of the IRES when the nucleotidic sequence of interest is operably linked downstream of the IRES, and the nucleotidic sequence encoding drug resistance cytidine deaminase (CD), a functional fragment or an analog thereof, being operably linked downstream of the IRES when the nucleotidic sequence of interest is operably linked upstream of the IRES.

The expression vector of the invention may further be flanked by retroviral long terminal repeat (LTR) sequence at 5′ and/or 3′ ends of the vector. The expression vector may be composed of DNA or RNA selected from the group consisting of eukaryotic, viral, adenoviral, adeno-associated, Simliclei, and Herpes simplex expression vectors.

The cells of the present invention may be selected from the group consisting of stromal, epithelial, fibroblasts, myoblasts, muscular, stem, progenitor, blood, and hematopoietic cells.

The nucleotidic sequence of interest may for example encodes a protein selected from the group consisting of cytokine, interleukin, growth hormones, hormones, blood factors, marker proteins, immunoglobulins, antigens, releasing hormone, anticancer product, antiviral product, antiretroviral product, an antisense, an antiangiogenic product, and a replication inhibitor.

The promoter may be a cytomegalo-virus (CMV) promoter.

In accordance with the present invention there is also provided an expression vector, wherein the nucleotidic sequence encoding for drug resistance cytidine deaminase (CD), a functional fragment or an analog thereof.

In accordance with the present invention there is provided a host cell transduced and/or transfected with the expression vector of the invention, and uses thereof in the preparation of a medicament for ex vivo treatment.

Also in accordance with the present invention, there is provided a method of identifying genetically transduced or transformed cells expressing a nucleotidic sequence of interest. The method comprises the steps of:

a) providing an expression vector for identifying transduced and/or transfected cells expressing a nucleotidic sequence of interest, the expression vector comprising:

a suitable promoter compatible with the transfected cells;

an internal ribosome entry site (IRES);

a marker nucleotidic sequence encoding for drug resistance cytidine deaminase (CD), a functional fragment or an analog thereof, operably linked to the IRES; and

a nucleotidic sequence of interest operably linked to the IRES;

wherein the nucleotidic sequence encoding drug resistance cytidine deaminase (CD), a functional fragment or an analog thereof, being operably linked upstream of the IRES when the nucleotidic sequence of interest is operably linked downstream of the IRES, and the nucleotidic sequence encoding drug resistance cytidine deaminase (CD), a functional fragment or an analog thereof, being operably linked downstream of the IRES when the nucleotidic sequence of interest is operably linked upstream of the IRES;

b) transducing and/or transfecting cells with the expression vector of step a);

c) culturing the cells of step b) under conditions suitable for the expression vector to express the marker nucleotidic sequence encoding the drug resistance cytidine deaminase, and the nucleotidic sequence of interest, the nucleotidic sequence of interest coding for a protein of interest; and

d) treating cells with cytosine nucleoside analogs;

wherein the cells living after the treating step d) are indicative that the cells are transduced and/or transfected and capable of expressing the nucleotidic sequence of interest.

For the purpose of the present invention the following terms are defined below.

The term “functional fragment” as used herein is intended to mean any portion of the gene encoding for a peptide or protein having the activity of the cytidine deaminase (CD).

The term “analogs” as used herein is intended to mean any functional modified form of the cytidine deaminase. The modification may become from a mutated or modified form of the nucleotidic sequence encoding CD.

### BRIEF DESCRIPTION OF THE DRAWINGS

FIG. 1 is a schematic illustration of the murine stem cell virus (MSCV) derived retroviral plasmids;

FIG. 2 is a schematic illustration of DNA integrated retrovector;

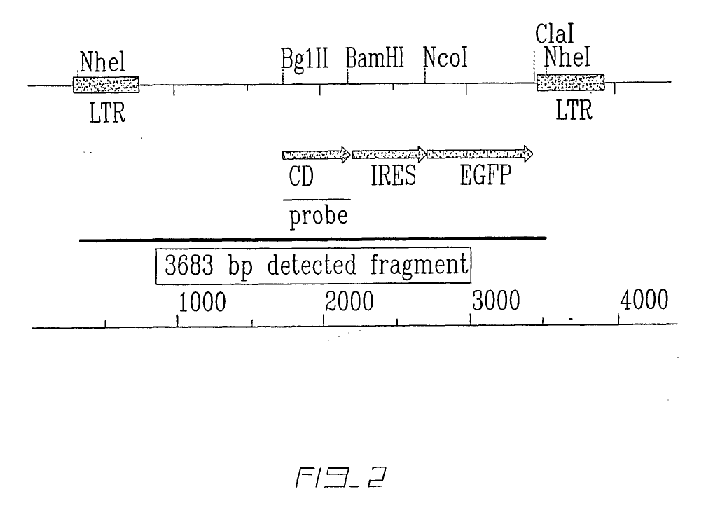

FIG. 3 illustrates southern blot analysis of CD-IRES-EGFP transduced A549 cells;

FIG. 4 illustrates the effect of ARA-C on the proliferation of genetically engineered A549 cells;

FIG. 5 illustrates the effect of dFdC on the proliferation of genetically engineered A549 cells;

FIG. 6 illustrates the resistance of CD gene-modified primary human lymphocytes to growth inhibition by ARA-C;

FIG. 7 illustrates the percent GFP positive cells assessed by flow cytometry analysis after in vitro enrichment of CD-IRES-EGFP transduced primary mouse marrow stromal cells;

FIG. 8 illustrates the flow cytometry analysis of one representative experiment demonstrating dose-dependent selective expansion of CD-IRES-EGFP transduced stromal cells; and

FIG. 9 illustrates the mean GFP fluorescence plotted against ARA-C concentration. Average±SEM (n=3).

### DETAILED DESCRIPTION OF THE INVENTION

In accordance with the present invention, there is provided a drug resistance gene (CD) that can serve as an in vitro or ex vivo positive selectable marker in genetically engineered cells, preferably primary autologous cells.

In one embodiment of the present invention, there is provided a human cytidine deaminase gene for ex vivo selective enrichment of genetically engineered primary cells, for cell and gene therapy purposes. The present invention also provide with an efficient selectable marker when linked to therapeutic exogenous nucleic acid sequences for treatment of genetic, malignant, immune, and viral diseases. For example, for patients implanted with ex vivo drug selected cells the method of the present invention would provide engrafted cells constituted entirely by genetically engineered cells, thus improving therapeutic outcome.

In another embodiment of the invention, there is provided a drug resistance gene with potential as a novel drug selectable marker. This drug resistance gene is the human cytidine deaminase (CD) gene. CD catalyses the deamination of cytidine or deoxycytidine to uridine or deoxyuridine, respectively and in addition, can deaminate cytosine nucleoside analogues causing their pharmacological inactivation. The CD drug resistance gene has recently shown promise for protecting normal hematopoietic cells from the dose-limiting myelotoxicity of anti-cancer drugs to thereby permit dose escalation for enhanced chemotherapeutic effectiveness. This chemoprotection approach using the human CD gene is the antithesis of a suicide gene therapy strategy utilising the prokaryotic cytosine deaminase. Unlike human CD or other optimal drug resistance genes, suicide genes code for non-mammalian enzymes, such as Escherichia coli cytosine deaminase and Herpes Simplex Virus Thymidine Kinase (HSV-TK), which are introduced into malignant cells to convert inactive prodrugs, such as 5-fluorocytosine and gancyclovir, respectively, into cytotoxic agents.

In the present invention, the human CD cDNA which consists of 910 bp encoding a 146 amino acid protein of 48.7 kilodaltons was cloned and expressed. The small size of the CD cDNA is a favourable feature endorsing its utility for gene therapy applications. It has demonstrated enhanced CD expression as well as cytosine nucleoside analogue drug resistance pursuant to retroviral gene transfer of the CD cDNA in mouse fibroblast and primary hematopoietic cells. Furthermore, CD proviral DNA persistence and long-term expression in various tissues has been determined in mice transplanted with CD gene-modified hematopoietic cells.

The human CD gene of the present invention possesses assets as a drug selectable marker. It is not a mutated variant nor prokaryotic, thereby indicating that drug selected CD gene-modified cells when implanted in vivo will not induce an immune response. Moreover, it has a small coding sequence and the agents that it efficiently confers resistance to, and which are to be utilised for ex vivo selection, are antimetabolites that will not cause DNA damage in CD-enriched cells. The main mechanism of action of ARA-C, an effective antileukemic drug, and dFdC, a promising antitumor agent, is via inhibition of DNA synthesis, whereas that of 5-AZA-CdR, which has shown antileukemic and interesting antitumor activity, is via inhibition of DNA methylation and ensuing activation of tumor suppressor genes. The human CD gene may serve as an ex vivo positive selectable marker with cytosine nucleoside analogues in genetically engineered primary autologous cells.

There is also provided with the invention an efficient in vitro drug selection of gene-modified primary murine marrow stromal cells. Bone marrow constitutes a source of stem cells for hematopoietic progenitors, and a provenance of mesenchymal stem cells or marrow stromal cells which have the ability to differentiate into several cell types including chondrocytes, osteoblasts, adipocytes, myoblasts, cardiomyocytes, and astrocytes. Marrow stromal is thus an autologous tissue that is an ideal target for cell and gene therapy strategies due to its capacity for self-renewal and differentiation.

Dominant selection of gene-modified marrow stromal cells ex vivo may allow a greater proportion of cells to express therapeutically relevant levels of the beneficial gene product in vivo thus enhancing clinical effectiveness.

In one embodiment of the present invention, there is provided bicistronic vector containing CD or the GFP reporter gene as marker operably linked to a gene encoding a protein of interest, generated retroparticles, and human A549 cells as well as primary human lymphocytes that can be transduced with these retrovirions in vitro and consequently may acquire the cytosine nucleoside analogue drug resistance or green fluorescence phenotype as indicators of efficient expression of the protein of interest. Primary murine marrow stromal cells may be also transduced and showed in vitro dose-dependent selection of CD gene-modified stromal cells using ARA-C.

Therefore, the introduction of a dominant selectable marker in marrow stromal allows selection and enrichment of genetically engineered cells ex vivo to ensure sufficient levels of the therapeutic protein for a favourable clinical outcome. The human CD gene possesses the potential to enhance the proportion of gene modified marrow stromal cells expressing a therapeutic transgene, such as type I collagen in children with osteogenesis imperfect. Furthermore, CD gene transfer may permit the ex vivo expansion of cells genetically engineered to express optimal amounts of therapeutic soluble proteins such as Factor VIII and Factor IX in hemophilia and Erythropoietin in hemoglobinopathies, such as β-thalassemia. Marrow stromal cells may thus serve as a gene delivery vehicle in diseases where clinical improvement is possible through the systemic secretion of a therapeutic gene product.

In one embodiment, target primary cells for selectable marker gene transfer may include bone marrow stromal cells and lymphocytes but also hematopoietic cells, myoblasts and/or fibroblasts. Gene transfer of human CD into hematopoietic stem cells and/or lymphocytes may be valuable in the treatment of disorders that afflict the hematopoietic system, such as adenosine deaminase deficiency and chronic granulomatous disease, as well as storage disorders such as Gaucher disease and Hunter syndrome. Additionally, CD for ex vivo selection may be used to augment the proportion of hematopoietic cells transduced with anti-HIV-I genes, such as RevMIO, and consequently give rise to an enriched population of mature T-lymphocytes and monocytic cells with high level antiviral gene expression.

Furthermore, the human CD gene may serve as a dominant selectable marker in cancer gene therapy applications employing a non-selectable therapeutic transgene such as a tumor-suppressor gene to inhibit tumor growth or a cytokine gene to strengthen the immune response against neoplastic cells. The human CD gene may also be utilised as a positive selectable marker for the therapy of autoimmune diseases such as arthritis, systemic lupus erythematosus, and colitis, by selectively enhancing cells genetically engineered to express regulatory cytokine genes such as IL-4 and IL-10, or inflammatory cytokine inhibitory genes such as IL-1, IL-2, TNFα, and IFNγ.

In contrast to selectable markers of prokaryotic origin, the CD gene, because of its human origin is not anticipated to be immunogenic in clinical applications, and as it is demonstrated in the present invention, has not impeded but enhanced the expression of a second transgene from a bicistronic retrovector.

Materials and Methods

Cell Lines

GP+E86 ecotropic and GP+envAM12 amphotropic retrovirus packaging cell lines, kindly supplied by A. Bank (Columbia University, New York) were maintained in Dulbecco's modified essential medium (DMEM) (Canadian Life Technologies, Burlington, Ontario) supplemented with 10% heat-inactivated foetal bovine serum (FBS) (Wisent Technologies, St. Bruno, Quebec) and 5 μg/ml gentamicin (Canadian Life Technologies).

The 293GPG retrovirus-packaging cell line, generously provided by R. C. Mulligan (Children's Hospital, Boston, Mass.) was grown in 293GPG media [DMEM supplemented with 10% heat-inactivated FBS, 0.3 mg/ml G418 (Mediatech, Herndon, Va.), 2 μg/ml puromycin (Sigma, Oakville, Ontario), 1 μg/ml tetracycline (Fisher Scientific, Nepean, Ontario), and 50 units/ml penicillin/streptomycin (Pen/Strep) (Wisent)].

A549 human lung carcinoma cells, obtained from American Type Culture Collection (ATCC), were maintained in RPMI 1640 medium (Canadian Life Technologies) supplemented with 10% heat-inactivated FBS.

NIH 3T3 mouse fibroblast cells, from ATCC, were cultured in DMEM with 10% FBS and 50 U/ml Pen/Strep. All cells were grown in a humidified incubator with 5% CO₂. (All cells were incubated at 37° C. with 5% CO₂).

Retroviral Vector Design and Virus-Producing Cell Line Generation

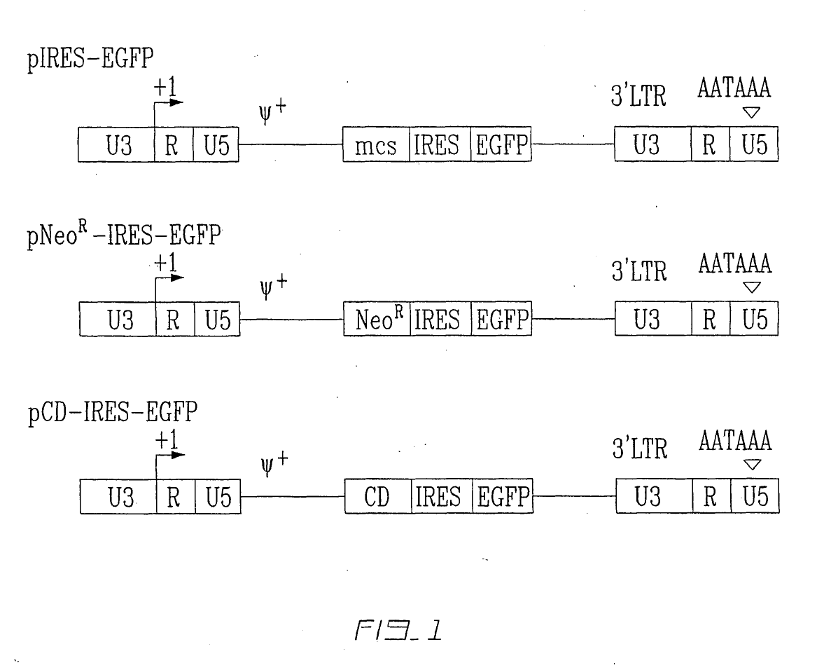

The murine stem cell virus (MSCV) derived retroviral vector pIRES-EGFP, a bicistronic construct comprising a multiple cloning site linked by an internal ribosomal entry site (IRES), to the enhanced green fluorescent protein (EGFP) reporter, was kindly provided by Robert Hawley. The retroviral vector pCD-IRES-EGFP (FIG. 1, pIRES-EGFP comprises a multiple cloning site (mcs) and the EGFP reporter gene parted by an internal ribosomal entry site (IRES). pNeo^{R}-IRES-EGFP is a derivative of pIRES-EGFP where the Neo^{R} gene is inserted in the mcs. Likewise, pCD-IRES-EGFP contains the human CD cDNA in the mcs upstream of the IRES) was synthesised by retrieving the human cytidine deaminase (CD) cDNA sequence by NcoI/Klenow and BamHI digest of the pMFG-CD construct, and ligating it with a XhoI/Klenow and BamHI digest of pIRES-EGFP. The retrovector pNeoR_IRES-EGFP, encompassing the neomycin phosphotransferase II drug resistance gene, was similarly constructed to serve as an additional control plasmid.

The pCD-IRES-EGFP vector (10 μg) was introduced into GP+E86 packaging cells by calcium phosphate transfection utilising the Cell Phect™ kit (Pharmacia) and cells were subsequently selected for 3 weeks in complete media supplemented with 2.5 μM cytosine arabinoside (ARA-C) (Upjohn, Don Mills, Ontario). The ensuing stable polyclonal producer cell population was used to supply virus for the transinfection (or transduction) of GP+envAM12 amphotropic packaging cells. Briefly, for 3 consecutive days, retrovirus-containing supernatant was harvested from subconfluent ecotropic producer cells, filtered with a 0.45 μm syringe mounted filter (Gelman Sciences, Ann Arbour, Mich.) and applied with 8 μg/ml Polybrenem (Sigma Chemical, St. Louis, Mo.) over target GP+envAM12 cells. Three days later, these amphotropic producers commenced a 2-week drug selection by culturing in media including 2.5 μM ARA-C, thence generating the polyclonal population GP+envAM12-CD-IRES-EGFP.

As a control, the GP+AM12-Neo^{R}-RES-EGFP polyclonal producer was created in an almost identical manner to that described above, the exception being that drug selection was performed with 400 μg/ml G418-containing media. In addition, GP+E86-Lac Z cells were generated by transinfection of the GP+E86 cell line with filtered retroviral supernatant from the 293GPG-LacZ producer (kind gift from R. C. Mulligan, Children's Hospital, Mass.) twice a day for 3 consecutive days. Moreover, utilising Lipofectamine™ (Gibco-BRL, Gaithesbug, Md.), the pantropic 293GPG packaging cell line is co-transfected with 5 μg pCD-IRES-EGFP and 70 ng pJ6ΩBleo graciously given by R. C. Mulligan (Children's Hospital, MA). These cells then underwent 4-week selection in 293GPG media supplemented with 100 μg/ml Zeocin™ (Invitrogen, San Diego, Calif.) consequently generating the stable polyclonal producer 293GPG-CD-IRES-EGFP. By this same approach, the control 293GPG-IRES-EGFP producer was also conceived. Flow cytometry analysis of cells for GFP expression was conducted using an Epics XL/MCL Coulter analyzer and gating live cells based on FSC/SSC profile. Retroparticles from virus producers were noted to be free from replication competent retrovirus (RCR) by GFP marker rescue assay employing supernatant from transduced target cells.

Titration of Retrovirus-Producers

To determine the titer of GP+E86 and GP+envAM12 producers, NIH 3T3 cells were utilised, whereas A549 cells were used for titering 293GPG producers. These target cells were plated at a density of 2 to 4×10⁴ cells per well in 6-well tissue culture dishes and the following day, cells from one test well were trypsinized and counted to ascertain the baseline cell number at time of virus addition. For transduction, serial dilutions of retroviral supernatants (0.01 to 100 μl in a final volume of 1 ml complete media, supplemented with 8 μg/ml polybrene, were placed over the adherent target cells. Flow cytometry analysis was realised 72 hours post-transduction to disclose the percentage of GFP-expressing cells. The titer was calculated utilising the equation below by considering the virus dilution that led to 10-40% GFP positive cells.

Titer (infectious particles/ml)=(% GFP positive cells)×(number of cells at initial virus exposure)/(volume of virus in the 1 ml applied to cells).

Transduction of Human A549 Cells, Analysis, and Drug Selection

Retroviral supernatant from 293GPG-CD-IRES-EGFP cells grown to confluence in 293GPG media devoid of tetracycline for over 72 hours to allow VSVG-pseudotyped retroparticle production were placed in a 25 cm² flask of subconfluent A549 cells with 8 μg/ml Polybrene™. This transduction procedure was executed once a day for 3 consecutive days yielding A549-CD-IRES-EGFP cells. As a control, A549 cells were likewise transduced with viral particles from 293GPG-IRES-EGFP producers hence giving rise to A549-IRES-EGFP cells. Five days following the last transduction round, flow cytometry analysis was performed to determine gene transfer efficiency and transgene expression as evaluated by GFP fluorescence. Stably transduced A549-CD-IRES-EGFP cells were subsequently expanded in complete media only, as well as in the presence of two different concentrations of ARA-C, 1 μM and 2.5 μM, for 12 days.

Southern Blot Analysis

Genomic DNA was isolated from CD-IRES-EGFP stably transduced A549 cells untreated or treated with ARA-C as well as from control cells, using a QIAGEN™ genomic DNA isolation kit. For Southern blot analysis, 5 μg of genomic DNA was digested with NheI and separated by agarose gel electrophoresis. Following UV photography with a fluorescent ruler, the gel was immersed in denaturing solution (0.5M NaOH; 1.5M NaCl) and then in neutralising buffer (0.5M Tris-HCl pH7; 1.5M NaCl, pH7), each for 45 minutes. The DNA in the gel was transferred onto a Hybond-N™ nylon membrane (Amersham, Oakville, Ontario) using 10×Standard Saline Citrate (SSC) for an approximately 48 hour downward transfer with the Turbo Blotter™ device (Schleicher & Shuell, Keene, N. H.). The membrane was then irradiated in a Bioslink™ UV linker with 0.3 J/cm² and hybridised in Express Hyb™ solution (Clontech) containing PCR-amplified ³²P-labeled cDNA for human CD. The membrane was washed and exposed using a Phosphor Imager™.

Growth Inhibition Assay (MTT) of Gene-Modified A549 Cells

Gene-modified A549 cells were plated in 96-well flat-bottom tissue culture dishes at a density of 1500 cells per well. Various concentrations of ARA-C or dFdC (Lilly Research Laboratories, Indianapolis, Ind.) were added to cells in a final volume of 100 μl RPMI/10% FBS. Cells were placed at 37° C. and 4 days later exposed to 20 μl of a solution containing 3-(4,5-deimethylthiazol-2-yl)-2,5-diphenyltetrazolium bromide, i.e. MTT, for 4 hours at 37° C., and subsequently to 100 μl of a solubilization buffer (50% N,N-dimethylformamide, 20% SDS) for approximately 15 hours. The optical density was then measured at 570 nm with reference at 630 nm using a multi-well plate reader, thus providing an assessment of the percentage of surviving cells based on the cleavage of MTT by the mitochondrial dehydrogenases of living cells to the detectable formazan. Percent cell survival was ascertained using the following equation:

$$\% cell survival = \frac{[ {OD}_{570 / 630} test well - O {D}_{570 / 630} media only well ]}{[ {OD}_{570 / 630} cells only well - O {D}_{570 / 630} media only well ]} \times 100.$$

CD Enzyme Assay

Stably transduced test and control A549 cells were assayed for CD enzyme activity. Concisely, these adherent cells (2-5×1 cells) were trypsinized, washed with phosphate-buffered saline (PBS) and resuspended in 5 mM Tris-HCl (pH 7.4) and 5 mM dithiothreitol. The cell suspension was freeze-thawed rapidly three times, centrifuged at high speed (12 000 rpm) for 15 minutes and the supernatant consisting of the cytosolic extract was collected. For CD enzyme assay, various dilutions of the cytosol were utilised in a reaction mixture with 50 mM Tris-HCl and 0.5 μCi ³H-cytidine (ICN Biomedicals, Irvine, Calif.). The reaction was allowed to proceed for 30 minutes at 37° C. and then stopped with cold HCl (0.001N). The mixture was subsequently poured onto Whatman™ P-81 phosphocellulose discs and the quantity of radioactivity bound to the discs determined by scintillation counting. Only cytosine nucleosides, and not the deaminated uracil nucleosides, are bound to the discs. One unit of enzyme activity was defined as the amount of enzyme that catalyses the deamination of one nmole of ³H-cytidine per minute at 37° C. Total protein concentration was determined using the Bio-Rad™ protein assay (Bio-Rad Laboratories, Mississauga, Ontario) with bovine serum albumin as the standard.

Transduction of primary human lymphocytes and growth suppression assay

Primary human lymphocytes were isolated from peripheral blood by Ficoll Hypaque™ centrifugation and plated at a cell density of ˜1×10⁶ cells/ml in RPMI supplemented with 10% FBS, 50 U/ml Pen/Strep, and 200 U/ml human IL-2. The next day, lymphocytes were activated with phytohemagglutinin (PHA) for 72 hrs and subsequently co-cultivated over subconfluent 293GPG-CD-IRES-EGFP or control 293GPG-IRES-EGFP virus producers tetracycline withdrawn three days earlier for optimal high titer virion generation. Lipofectaminel was added for a final concentration of 6 μg/ml, and co-cultivation permitted to proceed for 48 hours. Lymphocytes were then carefully collected and three days later plated at a density of 600 000 cells per well of 48-well tissue culture dishes in the absence or presence of various concentrations of ARA-C in a final volume of 800 μl) complete media. After a two week period, viable cells were enumerated by trypan blue exclusion and cell survival measured as such: % cell survival=(number of cells in test well/number of cells grown in media only)×100. (Flow cytometry analysis for CD3 expression was conducted and all cells confirmed to be lymphocytes.)

Harvest, Expansion and Transduction of Primary Mouse Bone Marrow Stoma

One female C57BI/6 mouse was sacrificed by CO₂ inhalation and bone marrow cells harvested by flushing the hind leg femurs and tibias with DMEM™ supplemented with 10% FBS and 50 U/ml Pen/Strep. Whole marrow was plated in 10 cm (diameter) tissue culture dishes and five days later, the nonadherent hematopoietic cells were discarded and the adherent bone marrow stromal cells cultured (for ˜3 months) at 37° C. with 5% CO₂. Supernatant containing virions from subconfluent GP+envAM12-CD-IRES-EGFP or from control GP+envAM12-Neo^{R}-IRES-EGFP producers was placed over marrow stromal cells plated at subconfluence. Transduction was carried out twice a day for three successive days in the presence of 6 μg/ml Lipofectamine™. Over 72 hours pursuing to the last transduction round, flow cytometry analysis for GFP expression was performed to evaluate the gene transfer efficiency.

In Vitro Selection and Enrichment Assay of CD Gene-Modified Marrow Stoma

CD-IRES-EGFP transduced stromal cells were mixed in various proportions with untransduced stromal cells and plated at a density of 25 000 cells/well of 6-well plates without ARA-C and with ARA-C at a final concentration of 2.5 μM. Drug exposure was ceased 7 days later, and cells expanded for an additional week in DMEMI with 10% FBS and 50U/ml Pen/Strep and analysed by flow cytometry analysis. Moreover, the cell combination where CD-IRES-EGFP modified stroma constituted 10% of total cell number was plated at 25 000 cells/well in 6-well dishes with a range of ARA-C concentrations. Pursuant to a 1-week drug exposure (preventing total confluence by cell passaging) and subsequent week of cell expansion, percent GFP positive cells and mean GFP fluorescence were evaluated by flow cytometry analysis.

Results

GFP Expression and Titer Determination of Virus-Producers

In order to evaluate gene transfer efficiency and transgene expression in stably transfected and transinfected virus producers, flow cytometry analysis for GFP expression was conducted. The percentage of GFP positive cells in the polyclonal producer populations GP+envAM12-CD-IRES-EGFP, GP+envAM12-Neo^{R}-IRES-EGFP, 293GPG-CD-IRES-EGFP, and 293GPG-IRES-EGFP were revealed to be 99.2, 100, 50.4, and 65.2, respectively, as compared to values of less than 3% for parental unaltered cells. To estimate the amount of infectious particles released by these cells, their retroviral supernatant was utilised in a titration assay and the extrapolated viral titers were 1.5×10⁶, 1.3×10⁶, 1×10⁶, and 0.5×10⁶ infectious particles per ml. (The titer of LacZ-GP+E86 virus producers estimated through X-gal staining of transduced NIH 3T3 cells, was ˜1.1×10⁵ infectious particles/ml.)

GFP Expression of CD Gene-Modified A549 Cells

Flow cytometry analysis was first executed on A549 cells succeeding their transduction and the percentage of cells expressing GFP was 95.9 for A549-CD-IRES-EGFP cells and 92.4 for A549-IRES-EGFP cells, in contrast to 0.2 for untransduced A549 cells.

Southern Blot Analysis

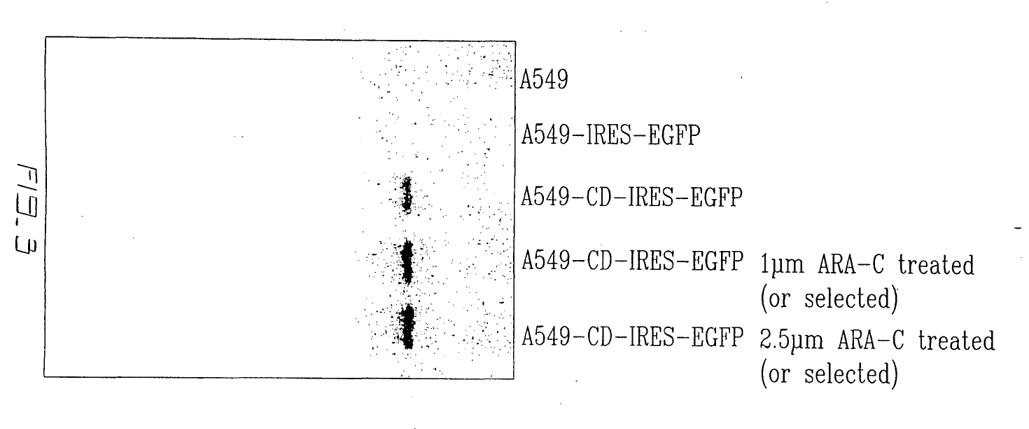

In order to demonstrate that the recombinant retroviral construct CD-IRES-EGFP did not sustain any rearrangements or deletions before integration as proviral DNA into the genome of transduced cells, Southern blot analysis was carried out on gene-modified A549 cells. This procedure revealed a DNA band corresponding to the 3683 bp fragment expected from NheI digest of integrated unrearranged CD-IRES-EGFP proviral DNA (FIG. 3). Genomic DNA from the indicated cell lines was digested with NheI, which cuts once in each flanking LTR, and fractionated on 1% agarose gel. Hybridisation of the blot with a ³²P-labeled CD cDNA probe permits detection of integrated, unrearranged proviral DNA of the predicted 3683 bp size. Molecular weights are indicated). In A549-CD-IRES-EGFP cells selected with 2.5 μM ARA-C, the DNA band detected was of higher intensity than that of cells exposed to 1 μM ARA-C, and notably more so than that of CD-transduced cells which were not drug selected (FIG. 3).

Expression of CD Enzyme Activity in Gene-Modified A549 Cells

To evaluate the CD activity in genetically engineered cells, enzyme assay was performed on the cytosolic extract of A549 cells transduced with CD-IRES-EGFP retroviral particles and on that of control cells, i.e. untransduced A549 cells, as well as A549 cells modified with IRES-EGFP virions. As detailed in Table 1, parental A549 cells as well as A549-IRES-EGFP cells showed low CD activity, specifically 0.29±0.07 and 0.53±0.07 Units/mg of protein, respectively. In contrast, the CD activity of A549-CD-IRES-EGFP cells was 515±31 Units/mg of protein, representing an over 1000-fold augmentation. CD enzyme assay was also conducted on the cytosol obtained from CD-IRES-EGFP transduced A549 cells exposed to 2.5 μM ARA-C for 12 days, and the CD enzyme activity measured was 524±41 Units/mg of protein. In Table 1, CD activity was measured in cytosolic extracts of the different cell lines. Units of enzyme activity are defined as nmoles of cytidine deaminated per minute.

TABLE 1
Cytidine deaminase (CD) activity in gene-modified A549 cells.

| Cell line | Enzyme Activity |
|---|---|
| A549 | 0.29 ± 0.07 |
| A549-IRES-EGFP | 0.53 ± 0.07 |
| A549-CD-IRES-EGFP | 515 ± 31 |
| A549-CD-IRES-EGFP | 524 ± 41 |
| (selected in 2.5 mM ARA-C) | |

Values represent average ± SEM, n = 4-12.

Growth Inhibition by ARA-C and dFdC of Gene-Modified A549 Cells (or: Drug Sensitivity of Gene-Modified A549 cells)

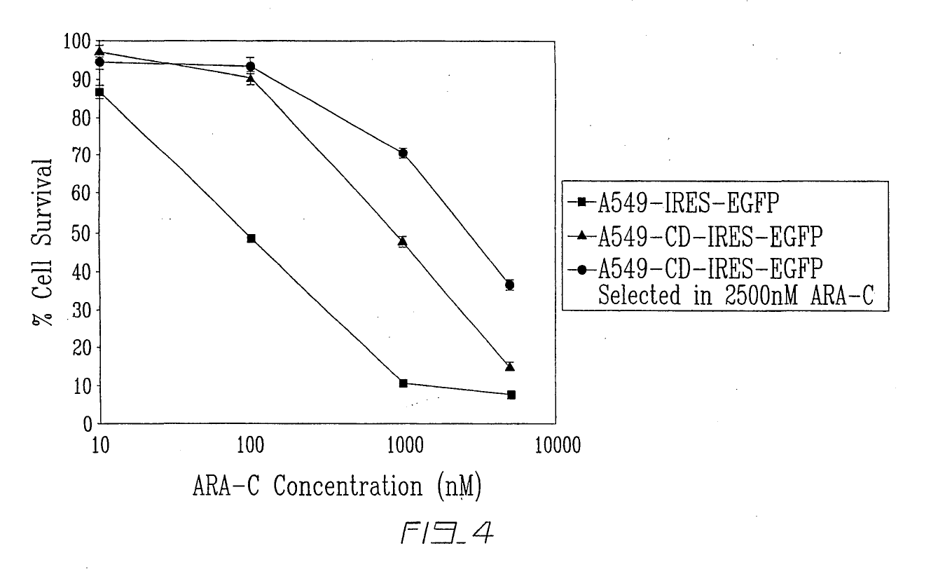

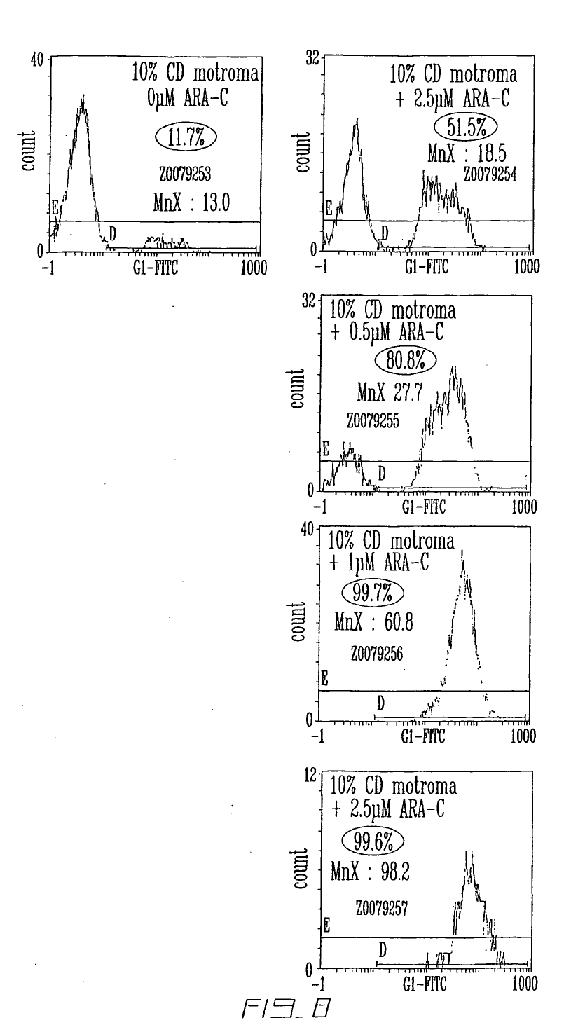

The sensitivity of genetically engineered A549 cells to the toxicity of cytosine nucleoside analogues ARA-C and dFdC was evaluated by MTT assay. As depicted in FIG. 8 (Parental A549 cells, A549 cells transduced with control IRES-EGFP retroparticles, and A549 cells transduced with CD-IRES-EGFP virions and expanded without ARA-C, with 1 μM ARA-C, or 2.5 μM ARA-C for 12 days, were subsequently exposed to ARA-C for 4 days and cell survival quantified by MTT assay. Percent survival is plotted against drug concentration (log scale). Average±SEM, n≧8), ARA-C at a concentration of 1 μM caused a substantial decrease in the % cell survival of untransduced A549 and A549-IRES-EGFP cells to values of 15.3±0.6 and 10.6±0.4, respectively. However, at this same drug concentration, survival by A549-CD-IRES-EGFP cells was over 3-fold greater at 47.4±1.9%. To demonstrate a relationship between in vitro drug selection pressure and resulting drug resistance magnitude, the sensitivity of the A549-CD-IRES-EGFP cells previously treated with 1 μM, as well as 2.5 μM ARA-C was also assessed. At the ARA-C concentration of 1 μM, the % survival of the 2.54M ARA-C-selected CD-modified cells was 70.7±1.3, thus significantly higher than the above-mentioned 47.4±1.9% cell survival of A549-CD-IRES-EGFP cells which had not undergone post-transduction drug selection (FIG. 4).

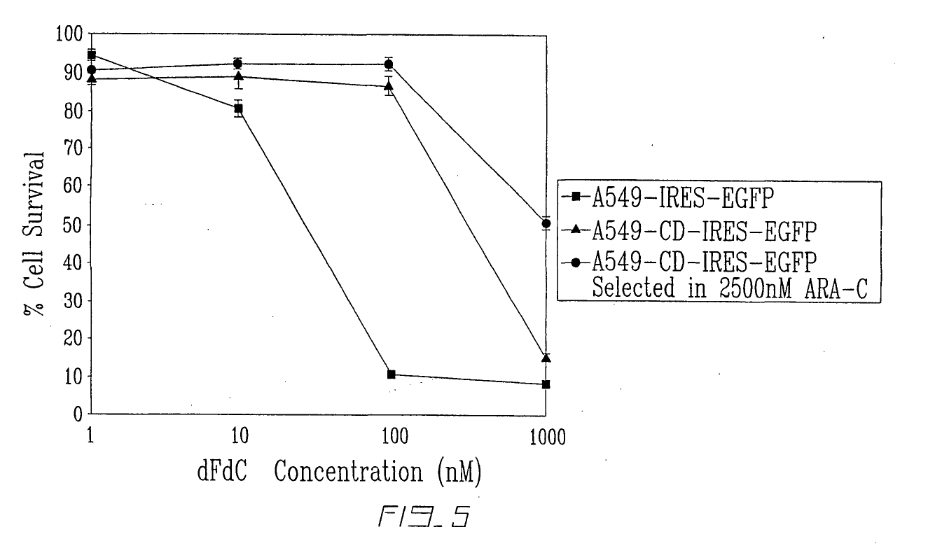

To ascertain that these CD gene-modified human cells are not resistant uniquely to ARA-C but have conjointly acquired cross-resistance to other cytosine nucleoside analogues, growth suppression by dFdC, was also evaluated. Treatment with 0.1M dFdC greatly suppressed cell survival of control A549 and A549-IRES-EGFP cells to 15.1±0.5% and 10.7±0.4%, respectively (Parental A549 cells, A549 cells transduced with control IRES-EGFP retroparticles, and A549 cells transduced with CD-IRES-EGFP virions and expanded without ARA-C, with 1 μM ARA-C, or 2.5 CM ARA-C for 12 days, were subsequently exposed to dFdC for 4 days and cell survival quantified by MTT assays. Percent survival is plotted against drug concentration (log scale). Average±SEM, n≧8). In contrast however, A549-CD-IRES-EGFP cells demonstrated considerably less drug sensitivity, with 86.4±2.4% survival at the same concentration of dFdC. Likewise, the 2.5 μM ARA-C selected CD-IRES-EGFP modified cells revealed at 0.1 μM dFdC, 92.1±1.7% cell survival. Furthermore, treatment with the highest concentration of 1 μM dFdC considerably reduced cell survival of unselected A549-CD-IRES-EGFP cells to 17.2±0.7% whereas the 2.5 μM ARA-C selected cells showed 53.3±1.8% survival (FIG. 5).

Effect of ARA-C on Growth of Genetically-Engineered Primary Human Lymphocytes

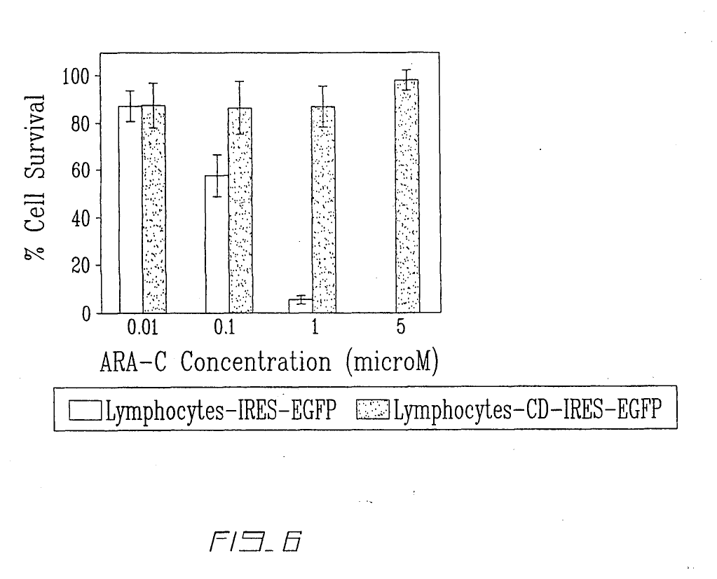

In vitro growth inhibition assay was performed to evaluate the sensitivity of gene-modified primary human lymphocytes to increasing concentrations of ARA-C. As illustrated in FIG. 6, treatment with 1 μM ARA-C practically abolished cell survival of human lymphocytes transduced with IRES-EGFP retrovirions (5.5±1.7% survival), whereas did not significantly affect that of lymphocytes transduced with CD-IRES-EGFP viral particles (87.5±7.2% cell survival). In addition, at the ARA-C concentration of 5 μM, growth of IRES-EGFP-modified lymphocytes was suppressed (0.1±0.1%), in vast contrast to CD-IRES-EGFP transduced lymphocytes which demonstrated virtually complete drug resistance (98.1+4.2% survival).

Ex Vivo Enrichment of CD Gene-Modified Primary Mouse Marrow Stromal Cells

Flow cytometry analysis was initially conducted on primary mouse bone marrow stromal cells 72 hours following their transduction, and the percentage of GFP positive cells was 98.4% for stroma engineered with CD-IRES-EGFP retroparticles and 96.3% for cells modified with NeoR_IRES-EGFP virus, as compared to 2.1% for untransduced stromal cells.

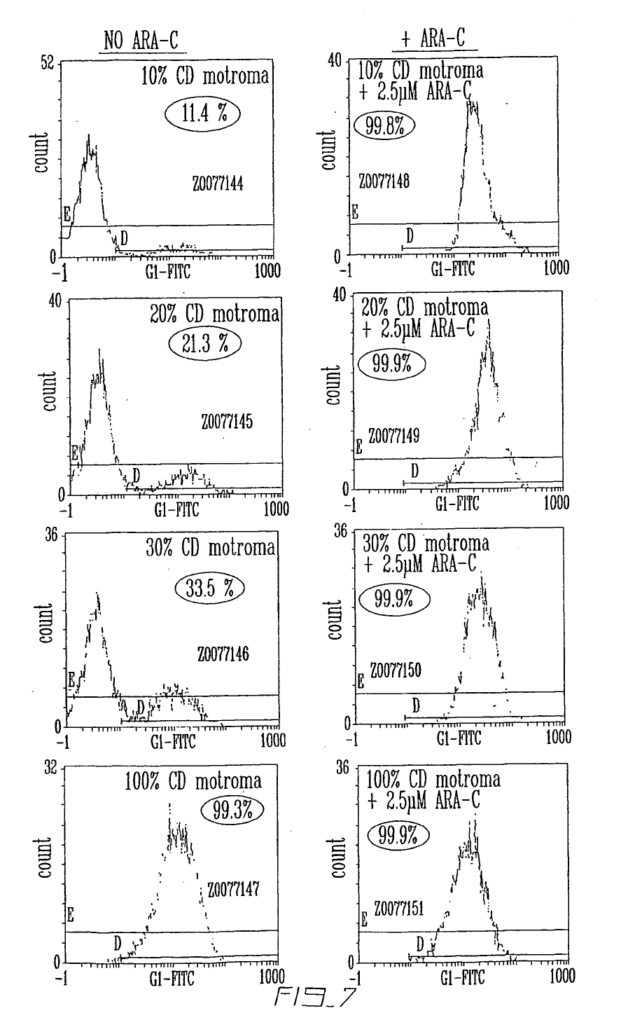

To determine if in vitro selection and enrichment of CD-expressing stroma can occur, CD-IRES-EGFP transduced stromal cells were mixed at 10, 20, 30, and 100% proportions with untransduced stroma, cultured in 2.5 μM ARA-C for 7 days, and expanded for one additional week. Subsequently, flow cytometry analysis revealed, as indicated in FIG. 7, selective expansion of GFP expressing cells to over 99% for all cell combinations exposed to ARA-C. (This ARA-C concentration of 2.5 CM was noted to completely eradicate untransduced as well as Neo^{R}-IRES-EGFP modified stroma). The average green fluorescence of CD-IRES-EGFP engineered stromal cells also rose ensuing drug selection (FIG. 7).

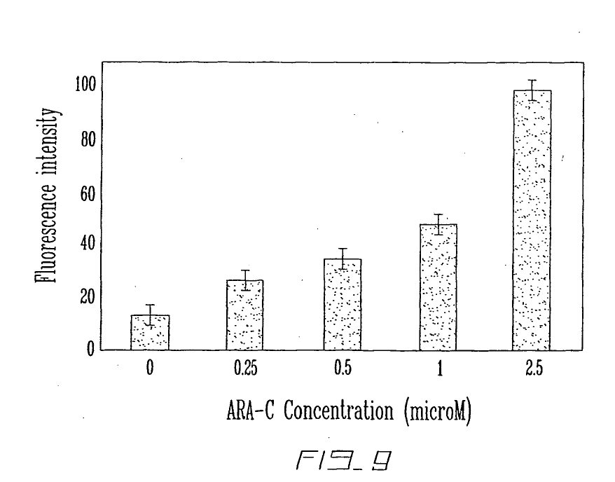

Moreover, to determine if in vitro enrichment of CD gene-modified marrow stroma is dose-dependent, a cell mixture consisting of 10% CD-IRES-EGFP vector-bearing stromal cells within a population of untransduced marrow stroma was exposed to increasing concentrations of ARA-C for one week and subsequently cultured for another week. Flow cytometry analysis revealed that the degree of selective expansion of the GFP positive cells was dependent on the dose of ARA-C (FIG. 6). As illustrated in FIG. 8, the 11.7% GFP+cells without ARA-C escalated to 51.5% and 99.6% when selected in 0.25 and 2.5 μM ARA-C, respectively (FIG. 8). Drug dose had an impact on the level of transgene expression, as perceived by the mean GFP fluorescence rising from 13.0±3.0 in the absence of ARA-C to 48.3±6.5 and 99.3±3.3 in cells selected in 1 and 2.5 μM ARA-C, respectively (FIG. 9).

In Vivo Engraftment of Ex Vivo Drug Selected Gene-Modified Stroma

To determine that ex-vivo drug selection of CD engineered stroma does not alter in vivo engraftment capacity, 10% CD-IRES-EGFP positive mouse stroma enriched to >99% by ARA-C exposure was thereafter gene-modified to also express β-galactosidase and implanted by intramuscular injection in immunocompetent syngeneic mice. As evidenced in the present experiment, sections of muscle harvested 2 weeks post-implantation contain engrafted ex vivo drug selected, transgene expressing cells.

Retroviral gene transfer permits stable and efficient integration of a foreign gene in the chromosomal DNA of the host cell and subsequently in all progeny cells. However, high level and long term expression of a beneficial exogenous gene is difficult to achieve in gene therapy studies. Nevertheless, gene therapy of many hereditary and acquired affliction commands that the desired therapeutic gene be efficiently translated in the majority of target cells. Accordingly, it is anticipated that if the genetically engineered cells represent only a small portion of the target cells, their outnumbering by the unaltered cells will ultimately ensue. One possible means to overcome this obstacle may be to expand the proportion of gene-modified cells in vitro through dominant selection relying on the co-expression of a drug resistance gene.

A bicistronic retroviral vector enclosing the human CD cDNA and the GFP reporter gene is generated. It is noted that its expression lead to functional levels of CD in genetically engineered human cell line A549, primary human lymphocytes, and primary murine bone marrow stromal cells. The convenience of this CD-IRES-EGFP retroviral construct (FIG. 1), where GFP represents the non-selectable therapeutic gene, was the accorded ability to track the gene-modified cells via the green fluorescence (emitted at 507 nm following blue light excitation at 488 nm) which was visualised by fluorescence microscopy and quantified by flow cytometry analysis. Therefore, GFP expression here reflected CD expression.

The CD-IRES-EGFP retrovector is integrated as an intact proviral DNA in the genome of transduced cells, since no rearrangements, nor deletions are revealed by Southern blot analysis of gene-modified cells (FIG. 3). The more intense cDNA signal detected with CD gene-modified A549 cells selected with 2.5 μM ARA-C, versus that with 1 μM ARA-C treated cells, or the weakest band seen with unselected cells, indicates an augmented copy number of the proviral sequences. This higher copy number suggests selection and enrichment of the CD positive cells with multiple integrants and/or amplification of the CD gene by ARA-C exposure of genetically-engineered cells. It was discovered that it is possible to increase CD expression via amplification of the proviral CD gene with ARA-C exposure of CD-transduced fibroblast cells.

Quantification of functional CD expression in CD-IRES-EGFP transduced A549 cells, as compared to corresponding control IRES-EGFP-modified cells, disclosed an over 1000-fold increment (Table 1). Thus, CD activity is markedly augmented in cells following stable transduction with CD-encoding retroparticles.

It has been established that CD gene transfer bestowed A549 cells with resistance to the growth inhibitory effect of ARA-C and dFdC, an over 10-fold increase in IC₅₀ noted for both antimetabolites (FIG. 3). Proliferation of CD-IRES-EGFP modified cells was considerably less inhibited by ARA-C and dFdC at concentrations that exhibited a pronounced cytotoxic effect on control IRES-EGFP altered A549 cells. In addition, selection of CD gene modified cells with ARA-C succeeding transduction further elevated their survival. These findings indicate that ARA-C treatment of cells ensuing transduction serves not only to eradicate the non-altered cells but also the low CD-expressers, thusly enriching the proportion of cells with the superior CD expression and consequently stronger drug resistance phenotype.

The ability to successfully genetically engineer and confer cytosine nucleoside analogue drug resistance to primary human cells is demonstrated, specifically in primary human lymphocytes which are transduced with CD-containing retrovirions and cultured in vitro in various concentrations of ARA-C. This is the first study to show CD gene transfer in primary human cells. Survival of CD-IRES-EGFP gene-modified lymphocytes is not significantly affected by ARA-C, even at a high dose which entirely suppressed cell survival by control IRES-EGFP transduced lymphocytes (FIG. 6). Therefore, the introduction of the CD cDNA into the drug sensitive primary human lymphocytes rendered these lymphocytes drug resistant and thus capable of surviving and even expanding in media comprising ARA-C concentrations that usually eradicate drug sensitive cells. These results propose the potential of the human CD gene for the positive selection of genetically engineered human lymphocytes, which may be a valuable tool in numerous cell and gene therapy studies. The transfer of therapeutic genes into lymphocytes may be used in the treatment of cancer, graft versus host disease (GVHD), AIDS, and autoimmune diseases. Enhancing with the CD, gene the population of transduced lymphocytes expressing a second non-selectable therapeutic gene may be beneficial, particularly in situations where poor transfer efficiency into lymphocytes is the limiting factor for successful gene therapy applications. For instance, ex vivo enrichment of gene-modified lymphocytes may be useful for the therapy of patients with adenosine deaminase deficiency and for the modulation of GVHD following hematopoietic cell transplantation for leukemia and lymphoma. Specifically, for controlling GVHD, the ex vivo enrichment using CD of donor T lymphocytes co-expressing the HSV-TK suicide gene may allow in vivo elimination with gancyclovir of all alloreactive donor cells following allogeneic bone marrow transplantation. Mouse marrow stromal cells transduced with CD-IRES-EGFP retroparticles and subsequently combined with untransduced stroma so as to constitute 10, 20, 30, and 100% of total cells were in all cases enriched with 2.5 μM ARA-C treatment to >99% gene-modified populations, as assessed by flow cytometry analysis for the linked GFP expression (FIG. 7). It was established that the selective in vitro expansion of transgene expressing cells was drug dose dependent, as observed by exposing the 10% CD-IRES-EGFP-containing stromal cells to a range of ARA-C concentrations (FIG. 6). It has been noted not only the dose-dependent enrichment of the retrovector positive stromal cells but also the dose-dependent enrichment of the cells possessing the strongest levels of transgene expression. Accordingly, as illustrated in FIG. 9, ARA-C dose escalation for in vitro selection of gene-modified stromal cells brought about expression augmentation, as evidenced by GFP fluorescence increments. Even though a high percentage of CD-IRES-EGFP positive cells were attained with the use of moderate drug concentrations, the more powerful doses of ARA-C selected the cells with the superior CD expression, thus strongest drug resistance phenotype. Amplification of the CD proviral DNA has also occurred with intensive ARA-C treatment of CD-IRES-EGFP transduced marrow stromal cells. Characteristics of an optimal drug resistance gene as a potential positive selectable marker comprise its effectiveness at imparting significant drug resistance, its small cDNA not imposing size constraints on the therapeutic gene included in the bicistronic vector, and very importantly its inability to raise an immune response.

Studies have demonstrated that genetically engineered primary cells, such as lymphocytes and hematopoietic cells can be dominantly selected not only through the concomitant expression of a drug resistance gene but also of a cell-surface reporter. Human and mouse hematopoietic cells have been gene-modified to express the cell surface protein human CD24 and were subsequently selected by fluorescence activated cell sorting (FACS). Retroviral gene transfer of the murine cell surface marker Heat Stable Antigen (HSA) or a truncated variant of human CD34 into human cells has also allowed the enrichment of gene-modified cells through FACS or immunoaffinity columns, respectively. In clinical applications it is very likely that the expression of a non-human selectable marker by transduced human cells will raise an immune reaction. Another selectable protein is the human low-affinity nerve growth factor receptor for which cell sorting is utilised to enrich genetically engineered cells based on cell-surface marking. However, with cell surface reporters the possibility exists that untransduced cells may be co-selected with gene-modified cells due to intercellular exchange of proteins on the surface of cells. This obstacle may be overcome with the use of cytoplasmic reporter proteins such as the green fluorescent protein (GFP). Many investigators have utilised the GFP reporter for the in vitro selection through FACS of transfected or transduced cells expressing high degree of fluorescence. Nevertheless, FACS may impose significant physical stress on gene-modified cells that may be detrimental particularly to sorted primary cells.

A further advantage of the human CD gene as a positive selectable marker is that the drugs that it confers resistance to, cytosine nucleoside analogues, and which are required for ex vivo selection, are antimetabolites that will not cause DNA damage in CD-enriched cells.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. An expression vector for identifying transduced and/or transfected cells expressing a nucleotidic sequence of interest, said expression vector comprising:
a suitable promoter;
an internal ribosome entry site (IRES);
a marker nucleotidic sequence encoding drug resistance cytidine deaminase (CD), a functional fragment or an analog thereof, operably linked to said IRES; and
a nucleotidic sequence of interest operably linked to said IRES;
wherein said marker nucleotidic sequence when detected in cells, indicates that said cells are transduced and/or transfected and capable of expressing said nucleotidic sequence of interest, said nucleotidic sequence encoding drug resistance cytidine deaminase (CD), a functional fragment or an analog thereof, being operably linked upstream of said IRES when said nucleotidic sequence of interest is operably linked downstream of said IRES, and said nucleotidic sequence encoding drug resistance cytidine deaminase (CD), a functional fragment or an analog thereof, being operably linked downstream of said IRES when said nucleotidic sequence of interest is operably linked upstream of said IRES.

2. The expression vector of claim 1, which is further, flanked by retroviral long terminal repeat (LTR) sequence at 5′ and/or 3′ ends of said vector.

3. The expression vector of claim 1, wherein said nucleotidic sequence of interest is endogenous or exogenous.

4. The expression vector of claim 1, which is composed of DNA or RNA.

5. The expression vector of claim 1, wherein said vector is selected from the group consisting of eukaryotic, viral, adenoviral, adeno-associated, Simliclei, and Herpes simplex expression vectors.

6. The expression vector of claim 1, wherein said cells are selected from the group consisting of stromal, epithelial, fibroblasts, myoblasts, muscular, stem, progenitor, blood, and hematopoietic cells.

7. The expression vector of claim 1, wherein said cells are autologous or heterologous cells of a patient.

8. The expression vector of claim 1, wherein said nucleotidic sequence of interest encodes for a protein selected from the group consisting of cytokine, interleukin, growth hormones, hormones, blood factors, marker proteins, immunoglobulins, antigens, releasing hormone, anticancer product, antiviral product, antiretroviral product, an antisense, an antiangiogenic product, and a replication inhibitor.

9. The expression vector of claim 1, wherein said promoter comprises a CMV promoter.

10. A host cell transduced and/or transfected with the expression vector of claim 1.

11. A method of identifying genetically transduced or transformed cells expressing a nucleotidic sequence of interest, said method comprising the steps of:
a) providing an expression vector as defined in claim 1;
b) transducing and/or transfecting cells with said expression vector of step a);
c) culturing the cells of step b) under conditions suitable for said expression vector to express said marker nucleotidic sequence and the nucleotidic sequence of interest, said nucleotidic sequence of interest coding for a protein of interest; and
d) treating said cells with cytosine nucleoside analogs;
wherein said cells living after the treating step d) are indicative that said cells are transduced and/or transfected and capable of expressing said nucleotidic sequence of interest.

12. The expression vector of claim 11, which is further flanked by retroviral long terminal repeat (LTR) sequence at 5′ and/or 3′ ends of said vector.

13. The method of claim 11, wherein said nucleotidic sequence of interest is endogenous or exogenous.

14. The method of claim 11, wherein said production of protein is performed in vitro, and/or in vivo.

15. The method of claim 11, wherein said expression vector is composed of DNA or RNA.

16. The method of claim 11, wherein said vector is selected from the group consisting of eukaryotic, viral, adenoviral, adeno-associated, Simliclei, and Herpes simplex expression vectors.

17. The method of claim 11, wherein said cells are selected from the group consisting of stromal, epithelial, fibroblasts, myoblasts, muscular, stem, progenitor, blood, and hematopoietic cells.

18. The method of claim 11, wherein said cells are autologous or heterologous cells of a patient.

19. The method of claim 11, wherein said patient is a human or an animal.

20. The method of claim 11, wherein said nucleotidic sequence of interest encodes for a protein selected from the group consisting of cytokine, interleukin, growth hormones, hormones, blood factors, marker proteins, immunoglobulins, antigens, releasing hormone, anticancer product, antiviral product, antiretroviral product, an antisense, an antiangiogenic product, and a replication inhibitor.

21. The method of claim 11, wherein said promoter comprises a CMV promoter.

22. A host cell transfected and/or transduced with the expression vector of claim 1.

23. Use of a host cells of claim 22 in the manufacture of a medicament for an ex vivo treatment.
